(19) United States
(12) Patent Application Publication
(10) Pub. No.: US 2014/0228664 A1
(43) Pub. Date: Aug. 14, 2014

(54) WEARABLE DEVICE FOR A USER

(71) Applicant: Ross Dominique Diaz Alcazar, Aurora, IL (US)

(72) Inventor: Ross Dominique Diaz Alcazar, Aurora, IL (US)

(73) Assignee: Ross Dominique Diaz Alcazar, Aurora, IL (US)

(21) Appl. No.: 13/844,893

(22) Filed: Mar. 16, 2013

Related U.S. Application Data
(60) Provisional application No. 61/754,606, filed on Feb. 14, 2013.

(51) Int. Cl. A61B 5/00

(52) U.S. Cl. CPC A61B 5/6813
USPC 600/382; 156/267

(57) ABSTRACT

The wearable device for sensing a body status is placed on the user. The wearable device includes a flexible printed circuit board and a semi-cured sheet encapsulatingly conforms to the shape of the flexible printed circuit board. The flexible printed circuit board includes a sensor for sensing body status and a communication device for sending the sensed signals to a server. The wearable device is made in the form of silicone semi-cured sheet.

### BACKGROUND OF THE INVENTION

1. Field of the Invention

The present invention generally relates to a wearable device for a user, and more particularly relates to a semi cured wearable device for sensing body status of a user.

2. Description of Related Art

Many devices are known in the art for monitoring the physical activities of the user, such as Nike Fuelband and Jawbone Up products. Most of these devices start with a bare flex assembled with IC chips, discreet components, connectors, large components, etc. This FPCBA (flexible printed circuit board assembly) is then either further sub-assembled into a carrier component or placed solely into an injection molding tool. A thermoplastic resin is then injected on and around the FPCBA to partially or fully encapsulate the FPCBA.

After injection, the resin is then fully cured and bonded permanently to the FPCBA. During the injection molding process, high velocity and high pressure of the resin is flowed over the FPCBA. This turbulence creates significant stress onto the flex potentially moving the flex out of alignment and original position. Additionally, the direct forces of the resin can create stress onto the solder joints and thus potentially break them. Therefore, there is a need of an ecpasulated semi-cured device for sensing body activities. Furthermore, the device for removing the liquid state of high injection pressure and replacing it with a semi-liquidous (gel) state of resin, rubber, silicone, and then apply the said gel to the FPCBA by a lamination process with a controlled force of application.

### SUMMARY OF THE INVENTION

In accordance with the teachings of the present invention an encapsulated semi-cured wearable device for sensing body status is provided.

An object of the present invention is to provide an encapsulated semi-cured wearable device for sensing a body status includes a flexible printed circuit board for sensing body status and first semi-cured sheet encapsulated on the top of flexible printed circuit board.

Another object of the present invention is a wearable device including a second semi-cured sheet encapsulated on the bottom of flexible printed circuit board.

Another object of the present invention is a wearable device including a first protective layer coupled with first semi-cured sheet and a second protective layer coupled with second semi-cured sheet.

### BRIEF DESCRIPTION OF DRAWINGS

FIG. 1 illustrates an exploded view of a wearable device in accordance with a preferred embodiment of the present invention;

FIG. 2 illustrates a flow process of assembling a wearable device in accordance with another preferred embodiment of the present invention;

FIG. 3a illustrates a perspective view of a wearable device on a user in accordance with an exemplary embodiment of the present invention; and

FIG. 3b illustrates a perspective view of a band containing a wearable device in accordance with another exemplary embodiment of the present invention.

### DETAILED DESCRIPTION OF DRAWINGS

While this technology is illustrated and described in a preferred embodiment, the wearable device may be produced in many different configurations, forms and materials. There is depicted in the drawings, and will herein be described in detail, as a preferred embodiment of the invention, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the technology described herein.

Reference will now be made in detail to several embodiments of the invention which are illustrated in the accompanying drawings. Wherever feasible and convenient, the same reference numerals are used in the figures and the description to refer to the same or like parts. The drawings are in a simplified form and not to precise scale. For purposes of convenience and clarity only, directional terms, such as top, bottom, left, right, up, down, over, above, below, beneath, rear, and front may be used with respect to the accompanying drawings. These and similar directional terms should not be strictly construed to limit the scope of the invention. In addition, words such as attached, affixed, coupled, connected and similar terms with their inflectional morphemes are used interchangeably, unless the difference is noted or made otherwise clear from the context. These words and expressions do not necessarily signify direct connections, but include connections through mediate components and devices.

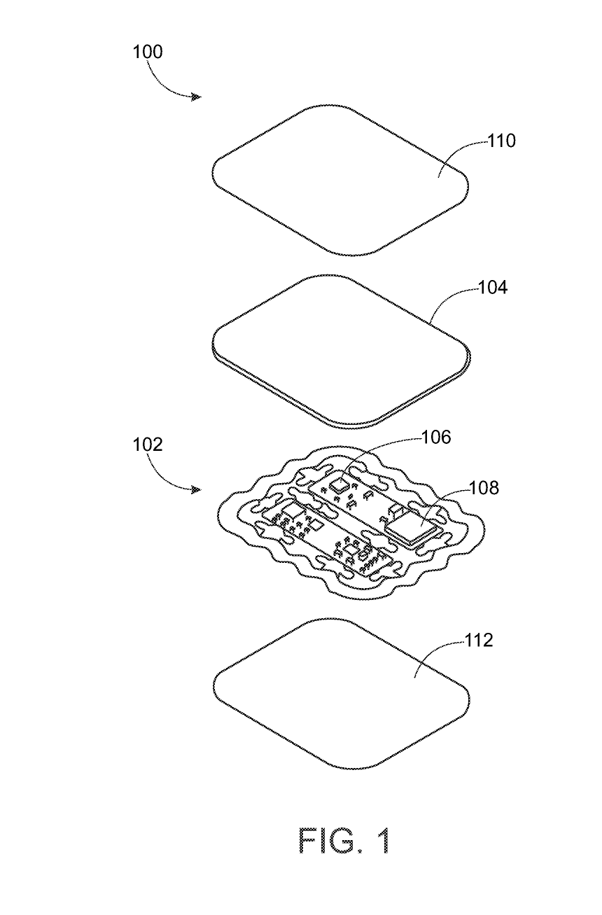

FIG. 1 illustrates an exploded view of the wearable device 100 for a user. The wearable device 100 includes a flexible printed circuit board 102 and a first semi-cured sheet 104 encapsulating the top side of the flexible printed circuit board 102. The flexible printed circuit board 102 includes a one or more sensors 106 for sensing body status of the user and one or more communication devices 108 for sending sensed signals from said one or more sensors to a server. Examples of communication devices include but not limited to bluetooth, zigbee, short range wireless, long range wireless etc.

The first semi-cured sheet 104 encapsulatingly conforms to the components of flexible printed circuit board 102 from the top side to form shape of flexible printed circuit board 102. In a preferred embodiment of the present invention, the first semi-cured sheet 104 is in a gel form.

In another embodiment of the present invention the wearable device 100 further includes a second semi-cured sheet encapsulatingly conforms to the components of flexible printed circuit board 102 from the bottom side to form shape of flexible printed circuit board 102. The second semi-cured sheet is in a gel form. In another preferred embodiment of the present invention the first and second semi-cured sheet is made from silicone.

As shown in FIG. 1, the wearable device 100 includes a first protective layer 110 coupled with the first semi-cured sheet 104 and a second protective layer 112 is coupled with the bottom side of the flexible printed circuit board 102. In another embodiment of the present invention the second protective layer 112 is coupled with the second semi-cured sheet (not shown in FIG. 1).

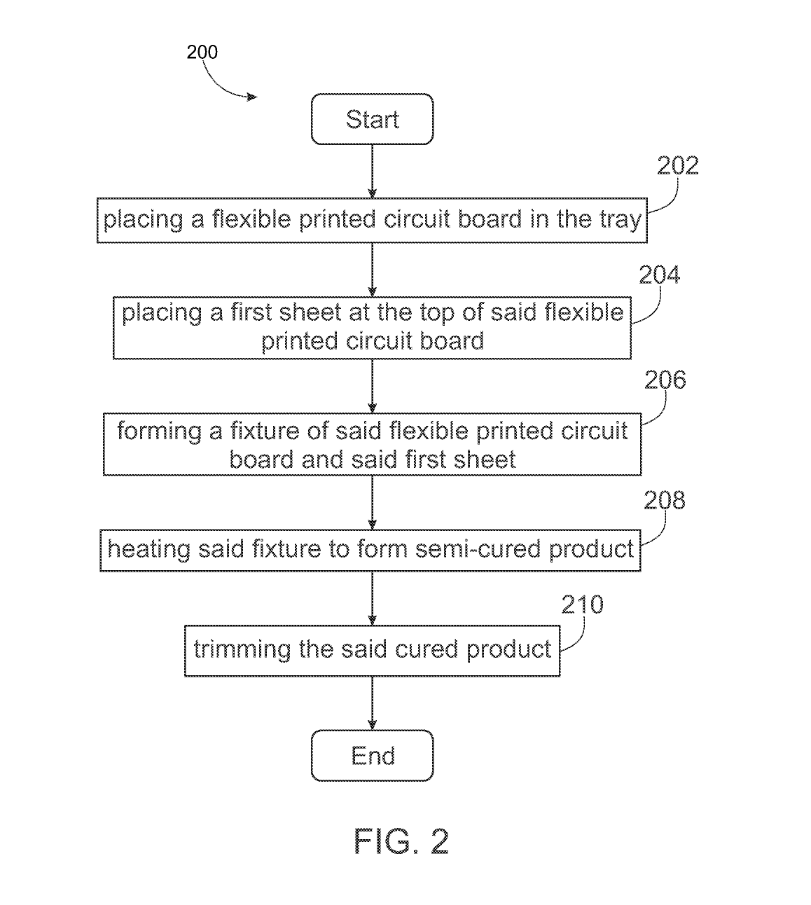

FIG. 2 illustrates a flow process 200 of assembling a wearable device in accordance with another preferred embodiment of the present invention. The method 200 initiates with a step 202 for placing a flexible printed circuit board in the tray. The step 202 is followed with a step 204 for placing a first sheet at the top of said flexible printed circuit board. The step 204 is then followed by a step 206 for forming a fixture of the flexible printed circuit board and the first sheet. The step 206 is then followed by a step 208 for heating the fixture to form a semi-cured product. The step 208 is then followed by a step 210 by trimming the first cured product. The semi-cured product is formed through silicone. It will be readily apparent to those skilled in the art noted that the heating and trimming may be performed by various devices without deviating from the scope of present invention.

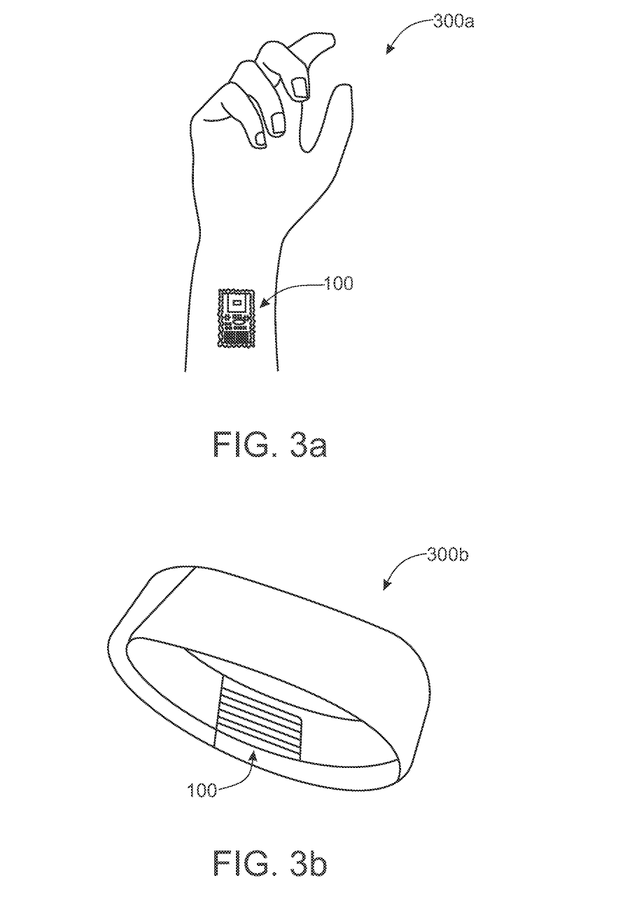

FIG. 3a illustrates a perspective view of wearable device 100 on a user's hand 300a in accordance with the exemplary embodiment of the present invention. However, those skilled in the art may place the wearable device 300a on various parts of the user's body such as knee, head, neck, heart, legs and feet without deviating from the scope of the present invention. FIG. 3b illustrates a band 300b containing the wearable device 100. The band 300b may be worn by a user in order to determine the activities of a user. In another embodiment of the present invention the wearable device may be used along with a watch or headband or knee band etc.

The wearable device offers various advantages such as in determining the UV and SPF sensing, EFG, EKG, skin hydration and firmness sensing, temperature and galvanic skin sensing, heart rate sensing, EMG, accelerometer and gyroscope sensing, sleep, emotion, gait and weight distribution sensing. However it is to be readily apparent to those skilled in the art that various other sensors may be used without deviating from the scope of the present invention.

The foregoing discussion discloses and describes merely exemplary embodiments of the technology described herein. One skilled in the art will readily recognize from such discussion and from the accompanying drawings that various changes, modifications and variations can be made therein without departing from the spirit and scope of the invention.

1. A wearable device for a user, said wearable device comprising:
a flexible printed circuit board comprising one or more sensors for sensing body status of said user and one or more communication devices for sending sensed signals from said one or more sensors to a server; and
a first semi-cured sheet encapsulatingly conforms to the top side of said flexible printed circuit board.

2. The wearable device according to claim 1 further comprises a second semi-cured sheet encapsulatingly conforms to the bottom side of said flexible printed circuit board.

3. The wearable device according to claim 1 wherein said first semi-cured sheet and said second semi-cured sheet is a silicon sheet.

4. The wearable device according to claim 1 wherein the said first and said second semi-cured sheet may be formed or pre-formed in a three dimensional geometry.

5. The wearable device according to claim 1 further comprises a first protective layer coupled with said first semi-cured sheet.

6. The wearable device according to claim 1 further comprises a second protective layer coupled with said second semi-cured sheet.

7. The wearable device according to claim 1 further comprises a second protective layer coupled with the bottom side of said flexible printed circuit board.

8. A wearable device for a user, said wearable device comprising:
a flexible printed circuit board comprising one or more sensors for sensing body status of said user and one or more communication devices for sending sensed signals to a server; and
a first semi-cured silicon sheet encapsulating the top side of said flexible printed circuit board.

9. The wearable device according to claim 7 further comprises a second-semi cured silicon sheet encapsulating the bottom side of said flexible printed circuit board.

10. The wearable device according to claim 7 further comprises a first protective layer coupled with said first semi-cured silicon sheet.

11. The wearable device according to claim 7 wherein the said first and said second semi-cured sheet may be formed or pre-formed in a three dimensional geometry.

12. The wearable device according to claim 7 further comprises a second protective layer coupled with said second semi-cured silicon sheet.

13. The wearable device according to claim 7 further comprises a second protective layer coupled with the bottom side of said flexible printed circuit board.

14. A method for assembling a wearable device, said method comprising the steps of:
placing a flexible printed circuit board in the tray;
placing a first sheet at the top of said flexible printed circuit board;
forming a fixture of said flexible printed circuit board and said first sheet;
heating said fixture to form a semi-cured product; and
trimming the said cured product.

15. The method for assembling a wearable device according to claim 12 further comprising the step of placing a second semi-cured sheet at the bottom of said flexible printed circuit board.

16. The method for assembling a wearable device according to claim 12 further comprising the step of placing a first protective sheet at the top of said first sheet.

17. The method for assembling a wearable device according to claim 12 further comprising the step of placing a second protective sheet at the bottom of said second semi-cured sheet.

18. The method for assembling a wearable device according to claim 12 wherein said first sheet and said second semi-cured sheet is comprised of silicone.
